(19) United States
(12) Patent Application Publication
(10) Pub. No.: US 2023/0227765 A1
(43) Pub. Date: Jul. 20, 2023

(54) THERMAL SEPARATOR APPARATUS FOR FLASK STIRRER AND METHOD OF USE

(71) Applicant: Genentech, Inc., South San Francisco, CA (US)

(72) Inventor: Chris Allen SEIDT, South San Francisco, CA (US)

(21) Appl. No.: 18/190,697

(22) Filed: Mar. 27, 2023

Related U.S. Application Data
(63) Continuation of application No. PCT/US2021/052881, filed on Sep. 30, 2021.
(60) Provisional application No. 63/085,343, filed on Sep. 30, 2020.

(51) Int. Cl. C12M 1/06; C12M 1/02; C12M 1/24; C12M 3/00

(52) U.S. Cl. CPC C12M 27/02; C12M 41/18; C12M 23/08; C12M 23/48

(57) ABSTRACT

Provided herein are systems and methods for thermally separating a flask and the base of an automatic flask stirring device. A thermal separator apparatus may be provided with a stand and a plurality of first standoffs. The stand may have a top surface configured to support a flask containing a substance to be stirred by a flask stirrer. The first standoffs may be located between the stand and a flask stirrer base, thereby creating a first air gap between the stand and the flask stirrer base. In some embodiments, the first air gap allows air to flow straight through the first air gap in a first direction.

### CROSS REFERENCE TO RELATED APPLICATIONS

This patent application is a continuation of International Application No. PCT/US2021/052881, titled “THERMAL SEPARATOR APPARATUS FOR FLASK STIRRER AND METHOD OF USE”, filed on Sep. 30, 2021, which claims priority to U.S. Provisional Patent Application no. 63/085,343, titled “THERMAL SEPARATOR APPARATUS FOR FLASK STIRRER AND METHOD OF USE”, filed on Sep. 30, 2020, each of which are herein incorporated by reference in its entirety.

INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

One of the challenges faced by biotechnology and pharmaceutical companies is to control the environmental conditions around cell cultures. Various mammalian cell cultures are useful during drug development, testing and production. For example, Chinese hamster ovary (CHO) cells are an epithelial cell line derived from the ovary of the Chinese hamster, often used in biological and medical research and commercially in the mass production of therapeutic proteins. They have found wide use in studies of genetics, toxicity screening, nutrition and gene expression, particularly to express recombinant proteins. CHO cells are often used as mammalian hosts for industrial production of recombinant protein therapeutics.

Another example of a laboratory media environment that needs to be tightly controlled is a media used to grow Escherichia coli (E. coli) bacteria. E. coli grows in a variety of defined laboratory media, such as lysogeny broth, or any medium that contains glucose, ammonium phosphate monobasic, sodium chloride, magnesium sulfate, potassium phosphate dibasic, and water.

To optimize growth in CHO and E. coli cultures, temperature, humidity, chemical concentrations and other factors need to be carefully managed. For example, these cultures do best when their temperature is held at 37° C. (98.6° F.). When the environmental temperature varies even slightly from this ideal temperature, sample growth or drug production can be slowed significantly, and the cultures can even die quickly.

CHO, E. coli and other cell cultures are often kept in flasks. In some implementations, flask sizes range between 100 ml and 6 liters. These sizes are often suitable not just for laboratory testing but also for production scale manufacturing. Frequent or constant stirring of the flask contents is often needed. To automate this task, an automatic stirrer may be employed. An automatic stirrer may have a stirring rod, pendulum or other type of agitator located inside the flask and physically, magnetically or otherwise coupled to a driving device located outside the flask. FIGS. 1 and 2 provide examples of prior art automatic stirrers. These are Variomag™ brand magnetic stirrers provided by Thermo Fisher Scientific Inc. of Waltham, Mass. USA. FIG. 1 depicts a single system that stirs one flask, and FIG. 2 depicts a multiple system that stirs four flasks simultaneously. In operation, the single system in FIG. 1 involves a stirring device 1, a stirring vessel or flask 2, a power supply 3, a secondary cable 4, a magnetic stirrer base 5 and a rotation speed control button 6. The base 5 of this device is 180 mm wide by 180 mm deep by 60 mm tall. The multiple system shown in FIG. 2 involves stirring devices 1, stirring vessels or flasks 2, a magnetic stirrer base 5′, a control cable 7, a power cable 8 and a control unit 9. The base 5′ of this device is 330 mm wide by 330 mm deep by 60 mm tall.

To aid in tightly controlling the environment in stirring vessel(s) 2, the vessel(s) may be placed inside an incubator. Typically, the vessel(s) 2 is/are placed inside the incubator along with a magnetic stirrer base 5, while the power supply 3 and or control unit 9 associated with the stirrer remains outside the incubator. Incubators can be provided with heating and cooling units to control temperature, active humidity controls, interior air circulation systems and HEPA air filtration. An example of a 232 liter capacity incubator suitable for this purpose is a Model No. 3307 Forma™ Steri-Cult™ CO₂ incubator provided by Thermo Fisher Scientific Inc. of Waltham, Mass. USA.

Even with all of the environmental controls outlined above, applicants of the present patent application have found it difficult to tightly control the environmental conditions in cell cultures, in particular, maintaining the temperature of the culture media at 37° C.

Accordingly, what is needed and is not provided by the prior art are improved systems and methods for maintaining desired environmental conditions in cell culture media. The innovations described herein solve these unmet needs and provide additional advantages.

### SUMMARY OF THE DISCLOSURE

According to aspects of the present disclosure, a thermal separator apparatus may be provided with a stand having a top surface configured to support a flask containing a substance to be stirred by a flask stirrer. The apparatus may also include a plurality of first standoffs configured to be located between the stand and a flask stirrer base. This arrangement creates a first air gap between the stand and the flask stirrer base that allows air to flow straight through the first air gap in a first direction.

In some embodiments of the above apparatus, the first air gap is configured to allow air to flow straight through the first air gap in a second direction, with the second direction being perpendicular to the first direction. The stand and or the first standoffs may include a material having a thermal conductively no higher than about 0.37 W/m·k. In some embodiments, the stand and the plurality of first standoffs are made from polyoxymethylene. In some embodiments, the stand does not have any vertical voids that connect the first air gap to the top surface of the stand.

In some embodiments, the apparatus may further include a platform having a top surface configured to support the plurality of first standoffs, and a plurality of second standoffs. The second standoffs may be configured to be located between the platform and a flask stirrer base. This arrangement creates a second air gap between the platform and the flask stirrer base that allows air to flow straight through the second air gap in a third direction.

In some embodiments having a platform and second standoffs, the second air gap is configured to allow air to flow straight through the second air gap in a fourth direction, with the fourth direction being perpendicular to the third direction. The platform and or the second standoffs may include a material having a thermal conductively no higher than about 0.37 W/m·k. In some embodiments, the platform and the plurality of second standoffs are made from polyoxymethylene. In some embodiments, the platform does not have any vertical voids that connect the first air gap to the second air gap.

In some embodiments having a platform and second standoffs, the first standoffs are laterally offset from the second standoffs. In some embodiments, the third direction is the same as the first direction and the fourth direction is the same as the second direction. The stand and the plurality of first standoffs may form a first stand set, and the apparatus may further include a plurality of substantially identical stand sets located on the top surface of the platform. In some embodiments, the apparatus comprises at least four stand sets. Each of the stands may include a plurality of different diameter recesses, each recess being configured to receive a different size flask.

In some embodiments, a minimum total horizontal cross-sectional area of the first standoffs does not exceed about 8% of a horizontal cross-sectional area of the stand. In some embodiments, a minimum total horizontal cross-sectional area of the second standoffs does not exceed about 29% of a horizontal cross-sectional area of the platform. In some embodiments, at least about 71% of the first air gap remains unblocked to straight airflow in the first direction. In some embodiments, at least about 44% of the second air gap remains unblocked to straight airflow in the third direction.

In some embodiments, the first standoffs are fastened to the platform with non-metallic fasteners. The second standoffs may be integrally formed with the platform. In some embodiments, the apparatus further includes a plurality of tabs projecting downwardly from outside edges of the platform that are configured to keep the apparatus centered on a flask stirrer base. In some embodiments a vertical distance between a bottom surface of any of the recesses and a bottom surface of the second standoffs does not exceed 5 inches. In some embodiments, a vertical distance between a bottom surface of any of the recesses and a bottom surface of the second standoffs does not exceed 1.5 inches.

In some embodiments, a thermal separator apparatus includes a plurality of stands, a platform, a plurality of first standoffs, a plurality of second standoffs and a plurality of tabs. In these embodiments, each stand has a top surface configured to support a flask containing a substance to be stirred by a flask stirrer. The platform has a top surface configured to indirectly support the plurality of stands. The plurality of first standoffs are configured to be located between an associated stand and the top surface of the platform. This arrangement creates a first air gap between each of the stands and the platform that allows air to flow straight through the first air gap in a first direction and in a second direction that is perpendicular to the first direction. The plurality of second standoffs are configured to be located between the platform and a flask stirrer base. This arrangement creates a second air gap between the platform and the flask stirrer base that allows air to flow straight through the second air gap in the first direction and the second direction. The plurality of tabs projects downwardly from outside edges of the platform and the tabs are configured to keep the apparatus centered on the flask stirrer base. The plurality of stands, the platform, the plurality of first standoffs and the plurality of second standoffs each comprise a material having a thermal conductively no higher than about 0.37 W/m·k. The plurality of first standoffs are each laterally offset from each of the second standoffs.

In some embodiments, a minimum total horizontal cross-sectional area of the first standoffs does not exceed about 8% of a horizontal cross-sectional area of the plurality of stands. In some embodiments, a minimum total horizontal cross-sectional area of the second standoffs does not exceed about 29% of a horizontal cross-sectional area of the platform. In some embodiments, at least about 71% of the first air gaps remain unblocked to straight airflow in the first and the second directions. In some embodiments, at least about 44% of the second air gap remains unblocked to straight airflow in the first and second directions.

According to aspects of the present disclosure, a method of automatically stirring the contents of a flask while closely maintaining the temperature of the contents includes the step of providing an automatic stirring device having a base. The method further includes providing a thermal separator apparatus as described above and placing it over the base of the stirring device. A flask is placed on at least one of the plurality of stands of the thermal separator apparatus. The at least one flask has contents to be stirred and a stirrer is magnetically coupled to the stirring device base. The method further includes locating the automatic stirring device, the thermal separator apparatus and the at least one flask in a temperature-controlled incubator and allowing air to circulate through the first and the second air gaps of the thermal separator apparatus.

### BRIEF DESCRIPTION OF THE DRAWINGS

The novel features of the disclosure are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1 is a perspective view showing a prior art single flask stirring device;

FIG. 2 is a perspective view showing a prior art multiple flask stirring device;

FIG. 3 is a perspective view showing a first exemplary embodiment of a thermal separator device constructed according to aspects of the disclosure;

FIG. 4 is side-elevation view showing the device of FIG. 3;

FIG. 5 is side cross-sectional view showing the device of FIG. 3;

FIG. 6 is top plan view showing the device of FIG. 3;

FIG. 7 is a perspective view showing a second exemplary embodiment of a thermal separator device constructed according to aspects of the disclosure;

FIG. 8 is a top plan view showing the device of FIG. 7;

FIG. 9 is a bottom view showing the device of FIG. 7;

FIG. 10 is a side-elevation view showing the device of FIG. 7; and

FIG. 11 is a side cross-sectional view showing the device of FIG. 7.

### DETAILED DESCRIPTION

According to aspects of the present disclosure, thermal separators disclosed herein are configured to provide thermal separation between a flask and a flask stirrer. These devices and the methods of their use are particularly beneficial in biotechnology and pharmaceutical experimentation and product manufacturing. As previously noted in the background section, even when flasks and flask stirrers are located in an incubator, it can still be difficult to tightly control the temperature of the contents of the flasks over time. Applicants of the present patent application have discovered that thermally isolating the flasks from the flask stirrers results in improved temperature control in the flasks. In particular, the temperature control is greatly increased when one or more levels of cross air circulation are provided by the thermal separators.

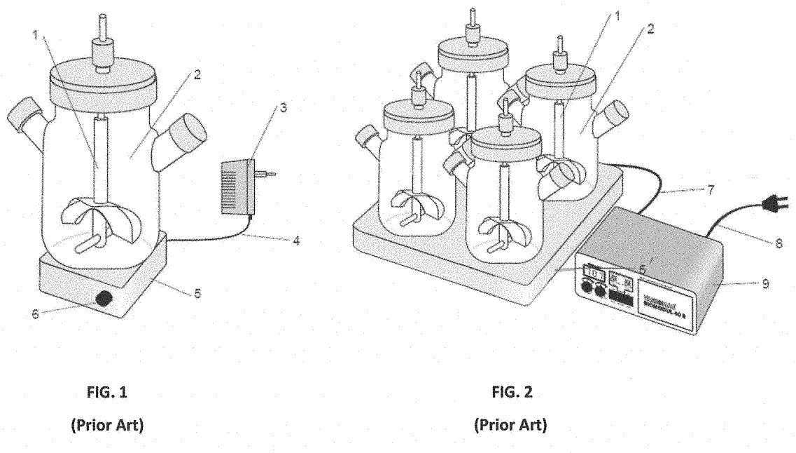

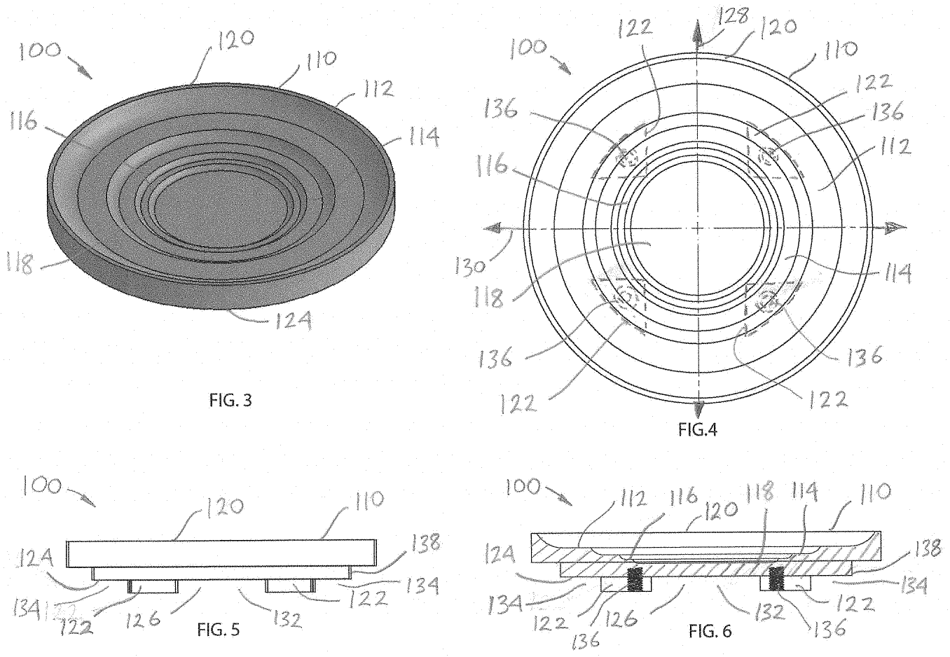

Referring to FIGS. 3-6, a first exemplary embodiment of a thermal separator device 100 constructed according to aspects of the disclosure is provided. A disk-shaped stand 110 may be provided with four recesses 112, 114, 116 and 118 in its top surface 120 as shown. Each recess may be progressively deeper and smaller in diameter. As such, each recess can serve to center and securely hold the bottom of a different size flask above a stirrer base, such as stirrer base 5 shown in FIG. 1 or stirrer base 5′ shown in FIG. 2. In this exemplary embodiment, recess 112 is configured to receive a 3000 ml flask, recess 114 is configured to receive a 500 ml flask, recess 116 is configured to receive a 250 ml flask, and recess 118 is configured to receive a 100 ml flask. In other embodiments, different size flasks can be accommodated and/or a different number of recesses can be provided, including zero or one recess.

Stand 110 may comprise a material having a thermal conductively no higher than about 0.37 W/m·k. In some embodiments, stand 110 is made from an acetal homopolymer or polyoxymethylene such as Delrin®.

As shown in FIGS. 4-6, a plurality of first standoffs 122 may be provided on the bottom surface 124 of stand 110. Standoffs 122 may be separate components that are attached to stand 110 with fasteners, adhesive or other known means, or they may be integrally formed with stand 110. In this exemplary embodiment, there are four standoffs 122, but other embodiments may have fewer or more first standoffs 122. In some embodiments, standoffs 122 are configured to be located between stand 110 and a top surface of a flask stirrer base 5 or 5′ (shown in FIGS. 1 and 2, respectively.) This arrangement creates a first air gap 126 between stand 110 and the flask stirrer base that allows air to flow straight through first air gap 126 in a first direction 128 (shown in FIG. 4.) The first air gap 126 in this exemplary embodiment also allows air to flow straight through the gap in a second direction 130 that is perpendicular to the first direction 128. Air can circulate through first gap 126 by natural convection, air currents created by an incubator enclosing thermal separator device 100 and or a dedicated fan system (not shown) associated with device 100. This gap and air circulation serve to inhibit heat from the flask stirrer base from reaching the flask through conduction, convection and or radiation.

In this exemplary embodiment, first air gap 126 includes two primary components in each direction 128 and 130: a central gap 132 located between first standoffs 122 and two side gaps 134 located between first standoffs 122 and the periphery of stand 110. In this embodiment, central gap 132 is about 0.25 inches tall (the height of first standoffs 122) and about 1.75 inches wide (the distance between first standoffs 122.) Side gaps are about 0.25 inches tall towards the center of stand 110, about 0.50 inches tall towards the periphery of stand 110 and have a maximum width of about 1.25 inches. The total width of first air gap 126 is about 6.0 inches (i.e. the diameter of stand 110), with about 4.25 inches (about 71%) of the width of first air gap remaining unblocked to straight airflow in first direction 128. The same is also true in second direction 130. In some embodiments, at least about 50%, about 70%, about 80% or about 90% of the first air gap 126 remains unblocked to straight airflow in a first direction 128.

First standoffs 122 may comprise a material having a thermal conductively no higher than about 0.37 W/m·k. In some embodiments, first standoffs 122 are made from an acetal homopolymer or polyoxymethylene such as Delrin®. This arrangement serves to inhibit heat from the flask stirrer base from reaching the flask through conduction.

In this exemplary embodiment, stand 110 is circular with an outer diameter of about 6.00 inches. In other embodiments (not shown), stand 110 may be square or another shape and or may have a size larger or smaller than about 6.00 inches. A stepped down portion 138 may be provided on the lower part of stand 110 and may have an outer diameter of about 5.00 inches. The major portion of stand 110 may be about 0.50 inches thick and the stepped down portion 138 may be about 0.25 inches thick, resulting in a total thickness of about 0.75 inches. In this exemplary embodiment, the total height of stand 110 and first standoffs 122 is about 1.00 inch.

In this exemplary embodiment, first standoffs 122 have a transverse cross-section that has the shape of a quarter-circle having a radius of about 0.875 inches. In this embodiment, the transverse cross-sectional area of each first standoff 122 is about 0.6 square inches, resulting in a total transverse cross-sectional area for all four standoffs 122 that is about 2.41 square inches. The bottom surface area of stepped down portion 138 of stand 110 is about 19.6 square inches, while the total bottom surface area of stand 110 is about 28.3 square inches. Accordingly, the total horizontal cross-sectional area of first standoffs 122 is about 12% of the horizontal cross-sectional area of the stepped down portion 138, and is about 8% of the horizontal cross-sectional area of stand 110 itself. In some embodiments, the first standoffs 122 do not have a constant horizontal cross-section along their vertical length. In these embodiments, the point of minimum horizontal cross-sectional area may be used in the above calculations. In some embodiments, a minimum total horizontal cross-sectional area of the first standoffs does not exceed about 4%, about 6%, about 8%, about 10% or about 12% of a horizontal cross-sectional area of stand 110.

In alternative embodiments, the first standoffs 122 may have a cross-section that is circular, oval, square, rectangular or other shape. The first standoffs 122 and or stand 110 may be provided with blind threaded holes 136 as shown for fastening stand 110 to the surface below it, such as with 5/16″ stainless-steel threaded fasteners (not shown.) In some embodiments, non-metallic fasteners are used to reduce heat conduction through first standoffs 122. In some embodiments, the fasteners are made of a homopolymer or polyoxymethylene such as Delrin®. In some embodiments, the surface directly below first standoffs when stand 110 is in use is a stirrer base 5 or 5′ (shown in FIGS. 1 and 2, respectively.) In other embodiments, additional components are located between first standoffs 122 and a stirrer base, an example of which is subsequently described in detail.

In this exemplary embodiment, the flat bottom portion of recess 112 has an outer diameter of about 4.946 inches, an inner diameter of about 3.950 inches and a depth of about 0.250 inches. The flat bottom portion of recess 114 has an outer diameter of about 3.517 inches, an inner diameter of about 2.975 inches and a depth of about 0.375 inches (below the top rim of stand 110.) The flat bottom portion of recess 116 has an outer diameter of about 2.763 inches, an inner diameter of about 2.512 inches and a depth of about 0.450 inches. The flat bottom portion of recess 118 has an outer diameter of about 2.296 inches and a depth of about 0.525 inches. Stand 110 has a minimum thickness of about 0.225 inches between the flat bottom portion of recess 118 and the bottom surface of stand 110. No vertical voids are included in this embodiment of stand 110 that might allow heat to travel directly vertically by convection through stand 110 to a flask resting thereon.

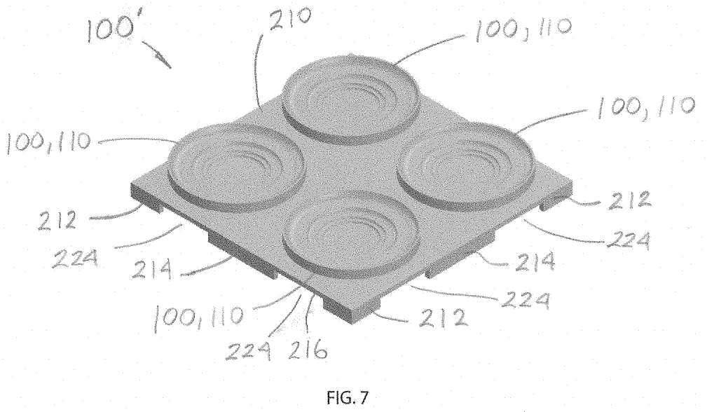

Referring to FIGS. 7-11, a second exemplary embodiment of a thermal separator device 100′ constructed according to aspects of the disclosure is provided. As best seen in FIG. 7, device 100′ is configured to fit over a multiple system stirrer base, such as base 5′ shown in FIG. 2. Device 110′ is configured to receive multiple flasks to be stirred, such as four flasks in this example. To receive the four flasks, four stands may be provided, such as stands 110 previously described in reference to FIGS. 3-6. Each stand 110 along with its associated first standoffs 122 may be referred to as a thermal separator or stand set 100. Device 100′ may further include a platform 210 configured to rest on top of a stirrer base 5′ shown in FIG. 2, between base 5′ and stand sets 100. Downwardly protruding corner tabs 212 and side tabs 214 may be provided under the outside edges of platform 210 to register device 100′ relative to the sides of the stirrer base and prevent it from moving from side to side, forward and back, and from rotating relative to the base. Stand sets 100 may be attached to the top of platform 210, as previously as subsequently described in more detail, to position each stand set 100 in three dimensions relative to the stirrer base for optimum magnetic coupling between stirring devices 1 and stirrer base 5′ (both shown in FIG. 2.) Features of stand sets 100 and platform 210 cooperate to provide multiple levels of thermal separation between a stirrer base and the flasks whose contents are being stirred.

Platform 210 may comprise a material having a thermal conductively no higher than about 0.37 W/m·k. In some embodiments, platform 210 is made from an acetal homopolymer or polyoxymethylene such as Delrin®.

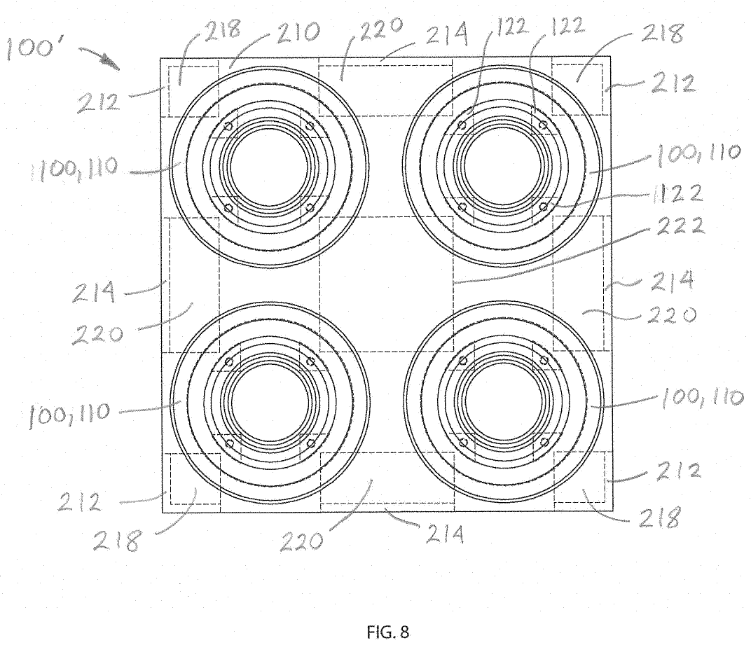

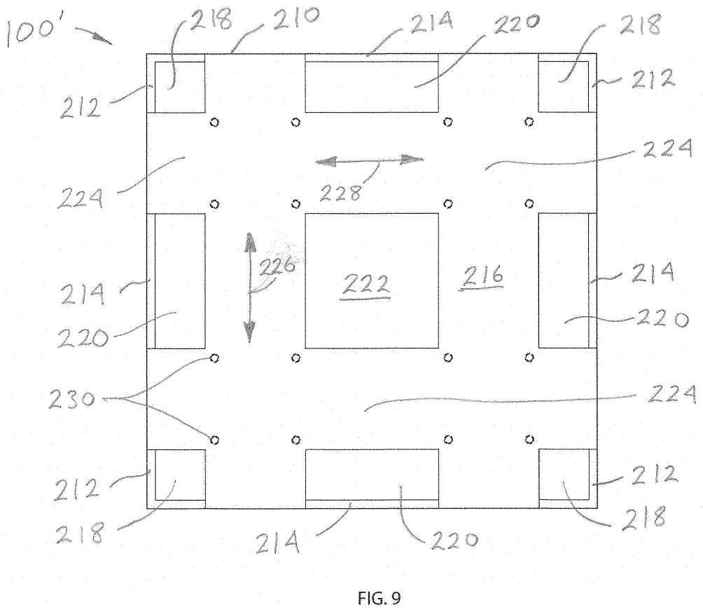

Referring to FIGS. 8 and 9, top and bottom views, respectively, of thermal separator device 100′are shown. The bottom of device 100′ may be provided with a second set of standoffs that each protrude downwardly from the bottom surface 216. The second set of standoffs may include four corner standoffs 218, four side standoffs 220 and one center standoff 222. Each of the second set of standoffs 218, 220 and 222 may protrude downwardly half as far as do the corner tabs 212 and side tabs 214. In this exemplary embodiment, the main portion of platform 210 is 0.25 thick, the second set of standoffs 218, 220 and 222 protrude downwardly 0.25 inches, and tabs 212 and 214 protrude 0.50 inches from bottom surface 216. Standoffs 218, 220 and 222, and or tabs 212 and 214 may be separate components that are attached to platform 210 with fasteners, adhesive or other known means, or they may be integrally formed with platform 210. In alternative embodiments, there may be fewer or more second standoffs 218, 220 and 222 and or tabs 212 and 214.

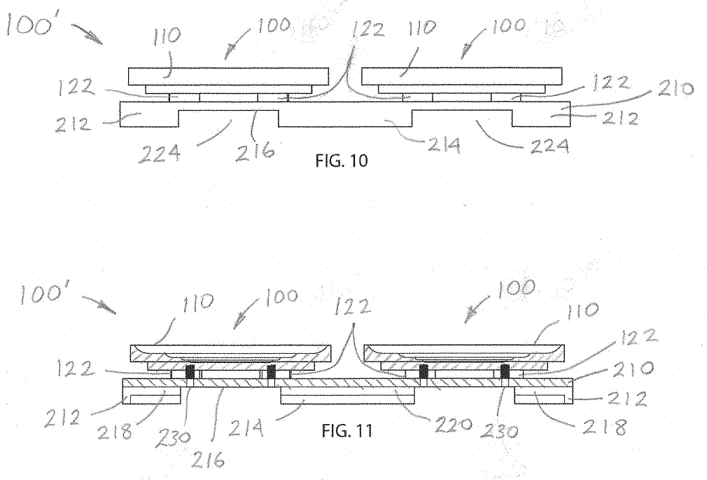

Downwardly protruding standoffs 218, 220 and 222 and tabs 212 and 214 leave a second air gap 224 for air to flow there between, and between the bottom surface 216 of platform 210 and the top surface of the flask stirrer base, as best seen in FIGS. 9 and 10. Second air gap 224 allows air to flow straight through second air gap 224 in a third direction 226, as shown in FIG. 9. The second air gap 224 in this exemplary embodiment also allows air to flow straight through the gap in a fourth direction 228 that is perpendicular to the third direction 226. In some embodiments including this exemplary embodiment, the third direction is the same as the first direction (shown in FIG. 4) and the fourth direction is the same as the second direction (also shown in FIG. 4.) Air can circulate through second gap 224 by natural convection, air currents created by an incubator enclosing thermal separator device 100′ and or a dedicated fan system (not shown) associated with device 100′. This gap and air circulation serve to inhibit heat from the flask stirrer base from reaching the flask(s) through conduction, convection and or radiation. It can also be seen in FIG. 10 that there are air gaps directly between adjacent stands 110 in this exemplary embodiment.

In this exemplary embodiment, second air gap 224 includes two channels in each direction 226 and 228. Both channels are about 0.25 inches tall (the height of second standoffs 218, 220 and 222) and about 3.00 inches wide (the distance between corner standoffs 218 and side standoffs 220, and also between side standoffs 220 and center standoff 222.) The total width of second air gap 224 is about 13.50 inches (i.e. the length of each side of platform 210), with about 6.00 inches (about 44%) of the width of second air gap remaining unblocked to straight airflow in third direction 226. The same is also true in fourth direction 228. In some embodiments, at least about 30%, about 50% or about 60% of the second air gap 224 remains unblocked to straight airflow in a third direction 226.

Second standoffs 218, 220 and 222 may comprise a material having a thermal conductively no higher than about 0.37 W/m·k. In some embodiments, second standoffs 218, 220 and 222 are made from an acetal homopolymer or polyoxymethylene such as Delrin®. This arrangement serves to inhibit heat from the flask stirrer base from reaching the flask through conduction.

In this exemplary embodiment, platform 210 is square with the inside edges of corner tabs 212 and side tabs 214 forming a 13.00 inch by 13.00 inch square that fits over the slightly smaller (12.99 inch by 12.99 inch) flask stirrer base. In other embodiments (not shown), platform 210 may be another shape and or may have a size larger or smaller than a flask stirrer base. The major portion of platform 210 may be about 0.25 inches thick, second standoffs 218, 220 and 222 may be about 0.25 thick, and tabs 212 and 214 may further extend about 0.25 inches, resulting in a total height of platform 210 of about 0.75 inches. In this exemplary embodiment, the total height of platform 210 and stand sets 100 is about 1.75 inches.

In this exemplary embodiment, corner standoffs 218 (not including corner tabs 212) have a transverse cross-section that is about 1.5 inches by about 1.5 inches, resulting in the four corner standoffs 218 having a total cross-sectional area of about 9.0 square inches. Side standoffs 220 (not including side tabs 214) have a transverse cross-section that is about 1.5 inches by about 4.0 inches, resulting in the four side standoffs 220 having a total cross-sectional area of about 24.0 square inches. Center standoff 222 has a transverse cross-section that is about 4.0 inches by about 4.0 inches, resulting in the center standoff 222 having a total cross-sectional area of about 16.0 square inches. Accordingly, the total transverse cross-sectional area for all nice standoffs 218, 220 and 222 is about 49.0 square inches. The bottom surface area of platform 210 is about 169.0 square inches. Therefore, the total horizontal cross-sectional area of second standoffs 218, 220 and 222 is about 29% of the horizontal cross-sectional area of platform 210. In some embodiments, the second standoffs 218, 220 and or 222 do not have a constant horizontal cross-section along their vertical length. In these embodiments, the point of minimum horizontal cross-sectional area may be used in the above calculations. In some embodiments, a minimum total horizontal cross-sectional area of the second standoffs does not exceed about 10%, about 20% about 40% or about 50% of a horizontal cross-sectional area of the platform 210.

In alternative embodiments, second standoffs 218, 220 and or 222 may have cross-sections that are circular, oval, square, rectangular or other shapes. Platform 210 may be provided with through holes 230, as best seen in FIGS. 9 and 11, for receiving fasteners therethrough, such as 5/16″ threaded fasteners (not shown) for attaching stand sets 100 to platform 210. As previously mentioned, non-metallic fasteners may be used. When the fasteners fill through holes 230, no vertical voids exist in this embodiment of platform 210 that might allow heat to travel directly vertically by convection through platform 210 toward flasks resting on stand sets 100. As shown in FIG. 8, first standoffs 122 may be laterally offset from second standoffs 218, 220 and 222 to further inhibit vertical heat transfer by conduction through thermal separator device 100′. In the current exemplary embodiment, edges of first standoffs 122 that are adjacent to edges of second standoffs 218, 220 and 222 are separated in a horizontal direction by about 0.08 inches.

In general, thermal separator device 100′ provides thermal separation between a flask stirrer base and flasks by providing two levels of transverse air gaps or channels, and two levels of vertical standoffs that are horizontally separated from one another. The unique combination of features provided by device 100′ inhibits heat transfer that might otherwise occur through conduction, convection and or radiation. The features disclosed herein can be extended to devices having more than two levels of air gaps and two levels of standoffs.

While exemplary embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the invention and that methods and structures within the scope of these claims and their equivalents be covered thereby.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the disclosure. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and/or methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. When a feature is described as optional, that does not necessarily mean that other features not described as optional are required.

The examples and illustrations included herein show, by way of illustration and not of limitation, specific embodiments in which the subject matter may be practiced. As mentioned, other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A thermal separator apparatus comprising:
a stand having a top surface configured to support a flask containing a substance to be stirred by a flask stirrer; and
a plurality of first standoffs configured to be located between the stand and a flask stirrer base, thereby creating a first air gap between the stand and the flask stirrer base that allows air to flow straight through the first air gap in a first direction.

2. The apparatus of claim 1, wherein the first air gap is configured to allow air to flow straight through the first air gap in a second direction, the second direction being perpendicular to the first direction.

3. The apparatus of claim 1, wherein the stand comprises a material having a thermal conductively no higher than about 0.37 W/m·k.

4. The apparatus of claim 1, wherein the first standoffs comprise a material having a thermal conductively no higher than about 0.37 W/m·k.

5. The apparatus of claim 1, wherein the stand and the plurality of first standoffs are made from polyoxymethylene.

6. The apparatus of claim 1, wherein the stand does not have any vertical voids that connect the first air gap to the top surface of the stand.

7. The apparatus of claim 1, further comprising:
a platform having a top surface configured to support the plurality of first standoffs; and
a plurality of second standoffs configured to be located between the platform and a flask stirrer base, thereby creating a second air gap between the platform and the flask stirrer base that allows air to flow straight through the second air gap in a third direction.

8. The apparatus of claim 7, wherein the second air gap is configured to allow air to flow straight through the second air gap in a fourth direction, the fourth direction being perpendicular to the third direction.

9. The apparatus of claim 7, wherein the platform comprises a material having a thermal conductively no higher than about 0.37 W/m·k.

10. The apparatus of claim 7, wherein the second standoffs comprise a material having a thermal conductively no higher than about 0.37 W/m·k.

11. The apparatus of claim 7, wherein the platform and the plurality of second standoffs are made from polyoxymethylene.

12. The apparatus of claim 7, wherein the platform does not have any vertical voids that connect the first air gap to the second air gap.

13. The apparatus of claim 7, wherein the first standoffs are laterally offset from the second standoffs.

14. The apparatus of claim 7, wherein the third direction is the same as the first direction and the fourth direction is the same as the second direction.

15. The apparatus of claim 7, wherein the stand and the plurality of first standoffs form a first stand set, and wherein the apparatus further comprises a plurality of substantially identical stand sets located on the top surface of the platform.

16. The apparatus of claim 15, wherein the apparatus comprises at least four stand sets.

17. The apparatus of claim 16, wherein each of the stands comprises a plurality of different diameter recesses, each recess being configured to receive a different size flask.

18. The apparatus of claim 1, wherein a minimum total horizontal cross-sectional area of the first standoffs does not exceed about 8% of a horizontal cross-sectional area of the stand.

19. The apparatus of claim 7, wherein a minimum total horizontal cross-sectional area of the second standoffs does not exceed about 29% of a horizontal cross-sectional area of the platform.

20. The apparatus of claim 1, wherein at least about 71% of the first air gap remains unblocked to straight airflow in the first direction.

21. The apparatus of claim 7, wherein at least about 44% of the second air gap remains unblocked to straight airflow in the third direction.

22. The apparatus of claim 1, wherein the first standoffs are fastened to the platform with non-metallic fasteners.

23. The apparatus of claim 7, wherein the second standoffs are integrally formed with the platform.

24. The apparatus of claim 7, further comprising a plurality of tabs projecting downwardly from outside edges of the platform and configured to keep the apparatus centered on a flask stirrer base.

25. The apparatus of claim 17, wherein a vertical distance between a bottom surface of any of the recesses and a bottom surface of the second standoffs does not exceed 5 inches.

26. The apparatus of claim 17, wherein a vertical distance between a bottom surface of any of the recesses and a bottom surface of the second standoffs does not exceed 1.5 inches.

27. A thermal separator apparatus comprising:
a plurality of stands, each stand having a top surface configured to support a flask containing a substance to be stirred by a flask stirrer;
a platform having a top surface configured to indirectly support the plurality of stands;
a plurality of first standoffs configured to be located between an associated stand and the top surface of the platform, thereby creating a first air gap between each of the stands and the platform that allows air to flow straight through the first air gap in a first direction and in a second direction that is perpendicular to the first direction;
a plurality of second standoffs configured to be located between the platform and a flask stirrer base, thereby creating a second air gap between the platform and the flask stirrer base that allows air to flow straight through the second air gap in the first direction and the second direction; and
a plurality of tabs projecting downwardly from outside edges of the platform and configured to keep the apparatus centered on the flask stirrer base,
wherein the plurality of stands, the platform, the plurality of first standoffs and the plurality of second standoffs each comprise a material having a thermal conductively no higher than about 0.37 W/m·k,
wherein the plurality of first standoffs are each laterally offset from each of the second standoffs.

28. The apparatus of claim 27, wherein a minimum total horizontal cross-sectional area of the first standoffs does not exceed about 8% of a horizontal cross-sectional area of the plurality of stands.

29. The apparatus of claim 27, wherein a minimum total horizontal cross-sectional area of the second standoffs does not exceed about 29% of a horizontal cross-sectional area of the platform.

30. The apparatus of claim 27, wherein at least about 71% of the first air gaps remain unblocked to straight airflow in the first and the second directions.

31. The apparatus of claim 27, wherein at least about 44% of the second air gap remains unblocked to straight airflow in the first and second directions.

32. A method of automatically stirring the contents of a flask while closely maintaining the temperature of the contents, the method comprising:
providing an automatic stirring device having a base;
providing a thermal separator apparatus according to claim 27 and placing it over the base of the stirring device;
placing a flask on at least one of the plurality of stands of the thermal separator apparatus, the at least one flask have contents to be stirred and a stirrer magnetically coupled to the stirring device base;
locating the automatic stirring device, the thermal separator apparatus and the at least one flask in a temperature-controlled incubator; and
allowing air to circulate through the first and the second air gaps of the thermal separator apparatus.
